(19) United States
(12) Patent Application Publication
(10) Pub. No.: US 2022/0267722 A1
(43) Pub. Date: Aug. 25, 2022

(54) Improved Retinal Organoids And Methods Of Making The Same

(71) Applicant: Newcells Biotech Limited, Newcastle upon Tyne (GB)

(72) Inventors: Valeria CHICHAGOVA, Newcastle upon Tyne (GB); Lyle ARMSTRONG, Newcastle upon Tyne (GB); Majlinda LAKO, Newcastle upon Tyne (GB)

(21) Appl. No.: 17/617,908

(22) PCT Filed: Jun. 8, 2020
(86) PCT No.: PCT/GB2020/051387

§ 371 Date: Dec. 9, 2021

(30) Foreign Application Priority Data
Jun. 10, 2019 (GB) 1908224.7

(51) Int. Cl. C12N 5/079; C12N 5/0793; A61P 27/00

(52) U.S. Cl. CPC C12N 5/0621; C12N 5/062; A61P 27/00; C12N 2506/02; C12N 2501/115; C12N 2501/165; C12N 2501/22; C12N 2501/727; C12N 2506/45

(57) ABSTRACT

The present invention relates to methods for making in vitro retinal cultures, tissue, or retinal organoids, from pluripotent cells as well as the improved synthetic retinal tissue and retinal organoids themselves. It also relates to retinal organoids that replicate in vitro many characteristics of the retina (e.g., human or mammalian), and methods of using this retinal organoid to study disease and to identify therapeutic agents for the treatment of retinal diseases and disorders.

### TECHNICAL FIELD

The present invention relates to methods for making in vitro retinal cultures, tissue, or retinal organoids, from pluripotent cells as well as the improved synthetic retinal tissue and retinal organoids themselves. It also relates to retinal organoids that replicate in vitro many characteristics of the retina (e.g., human or mammalian), and methods of using this retinal organoid to study disease and to identify therapeutic agents for the treatment of retinal diseases and disorders.

### BACKGROUND

According to NHS England, complete or partial vision loss affects around 2 million people in the UK alone, this represents a significant burden on the healthcare system and economy. Some of the most common causes of blindness are as a result of photoreceptor death. Currently, there are no treatments capable of replacing photoreceptor cells. Retinal degeneration is described as the destruction or deterioration of the retina caused by progressive and eventual death of the retinal cells. The retina is the sensory membrane or tissue that lines the inner surface of the back of the eyeball. It's composed of several layers, including one that contains specialized cells called photoreceptors. Retinal diseases vary widely, but most of them cause visual symptoms.

The disorders associated with retinal degeneration are collectively termed as retinal degenerative disorders and include prevalent retinal degenerative diseases such as diabetic retinopathy, retinopathy of prematurity, macular degeneration, Usher syndrome, Stargardt disease and Retinitis Pigmentosa.

In order to develop efficient therapies, it is vital that researchers have access to suitable model systems in which to test developing compounds and to study different diseases such as age-related macular degeneration (AMD) and inherited retinal dystrophies (IRDS).

Organoids are self-organising, 3D cell cultures which are valuable in many applications such as; drug screening, toxicity, disease modelling, and potentially in regenerative medicine. Organoid cultures have been described modelling different organs and tissues such as retina, brain and kidney.

Organoids can be derived from isolated primary progenitor cells or pluripotent stem cells which are directed towards differentiation pathways to yield the desired cell types. There is much interest in further developing organoids to improve their likeness to the in vivo organs and tissues which they are designed to model. The closer an organoid model is to the in vivo organ or tissue, the more effective it is when assessing toxicity and viability of therapeutics, for example.

Retinal organoid models have been developed which are derived from pluripotent stem cells (PSCs) such as in the protocol described in Nakano et al. (2012) whereby retinal organoids were derived from human PSCs, the resulting organoids are highly organised 3D structures but still do not fully represent the in vivo retina.

The in vivo retina is a complex organ comprising many neuronal cell types such as; retinal ganglion cells, photoreceptor cells, bipolar cells, horizontal cells and amacrine cells as well as other cell types such as immune cells and epithelial cells. In order to best recapitulate the naturally occurring retina, in vitro models should comprise different cell types.

### SUMMARY OF THE INVENTION

According to a first aspect of the present invention there is provided an improved pluripotent stem cell-derived, in vitro generated, retinal tissue comprising retinal cells and microglial cells.

Advantageously, by co-culturing microglial and/or microglial progenitor cells along with a retinal-like organoid tissue to form the improved retinal organoid, the resulting product is a synthetic retinal organoid that reacts to external factors in a manner more accurately reflects that of naturally occurring retinal tissue making it particularly useful for therapy or screening.

Most preferably the retinal tissue is a three-dimensional retinal-like organoid.

Advantageously, the synthetic retinal tissue is organised into a three-dimensional retinal organoid that has a layered structure which resembles a naturally occurring retina.

Preferably the improved three-dimensional retinal-like organoid comprises microglial cells as well as a plurality of cells selected from photoreceptor cells (PRCs), amacrine cells, muller glia, horizontal cells, bipolar cells and retinal ganglion cells.

Preferably the improved three-dimensional retinal-like organoid comprises a retinal pigment epithelium (RPE) layer.

Retinal pigment epithelium (RPE) layer, photoreceptor cells (PRCs), muller glia, horizontal cells, bipolar cells, retinal ganglion cells and microglial cells are identified as they resemble naturally occurring cells of the same type visually, functionally and physiologically for example in their expression patterns and reactions to external agents.

Preferably the pluripotent stem cells are induced pluripotent stem cells.

Most preferably the induced pluripotent stem cells (iPSCs) from which the tissue or organoid is derived is a mammalian induced pluripotent stem cell, more preferably a human induced pluripotent stem cell (hiPSC).

Optionally the induced pluripotent stem cells (iPSCs) are derived from a patient without any known genetic disorder i.e. a ‘healthy’ patient.

Optionally the induced pluripotent stem cells (iPSCs) are derived from a patient with a known genetic disorder.

Advantageously, by using induced pluripotent stem cells derived from a patient with a known genetic disorder a disease model organoid can be produced.

Preferably the organoid comprises cells expressing one or more retinal cell markers and a cell expressing one or more microglial markers.

Optionally the retinal cell markers are AP-2α, HuC/D, Prox1, Recoverin and/or CRX.

Optionally the expressed microglial cell markers are CX3C chemokine receptor 1 (CX3CR1) and/or Ionized calcium binding adaptor molecule 1 (IBA1). The CD14 marker may also be expressed in conjunction with the above.

CX3CR1 is also known as the fracalkine receptor or G-protein coupled receptor 13 (GPR13).

According to another aspect of the present invention, there is provided a method for obtaining retinal tissue or a retinal organoid comprising the steps of;

co-culturing the microglial or microglial progenitor cells and the retinal organoid under conditions which allow the microglial cells to integrate into the organoid.

Preferably the method includes the steps of;

obtaining a population of microglial cells and/or microglial progenitor cells;
obtaining a pluripotent stem cell derived retinal organoid; and
co-culturing the microglial cells and the retinal organoid under conditions which allow the microglial cells to integrate into the organoid.

Preferably, the obtained population of microglial or microglial progenitor cells has >40% cells positive for CD14 and/or CX3CR1.

Preferably the step of obtaining a population of microglial cells includes differentiating pluripotent stem cells into hematopoietic progenitor cells and then to microglial progenitors or microglial-like cells.

Preferably, when obtaining a population of microglial cells or microglial progenitor cells they are induced pluripotent stem cell derived microglial cells.

Preferably the pluripotent stem cell derived retinal organoid has a layered structure which resembles a naturally occurring retina.

Preferably the pluripotent stem cell derived retinal organoid comprises a plurality of cells selected from photoreceptor cells (PRCs), amacrine cells, muller glia, horizontal cells, bipolar cells, retinal ganglion cells and microglial cells. It may also comprise a retinal pigment epithelium (RPE) layer.

Preferably the pluripotent stem cell derived retinal organoid is positive for AP-2α, HuC/D, Prox1, Recoverin and/or CRX.

Preferably, prior to co-culturing the microglial cells and the retinal organoid, the microglial cells are left to mature for 3 or more days, preferably 5 or more days, most preferably 7 days.

Preferably, when co-culturing the microglial cells and the retinal organoid, the organoid is <250 days old, more preferably <200 days old and yet more preferably <150 days old. Most preferably the organoid is approximately 90-100 days old.

Preferably, when co-culturing the microglial cells and the retinal organoid, the microglial cells are plated at a density of <10000 cells per organoid, preferably the microglial cells are plated at a density of <8000 cells per organoid, most preferably the microglial cells are plated at a density of approximately 5000 cells per organoid.

Preferably, when the microglial cells and the retinal organoid are co-cultured for greater than 7 days, preferably greater than 10 days, optionally 14 days or more, preferably 14 days.

Yet more preferably, the microglial cells and the retinal organoid are co-cultured for greater than 55 days.

According to another aspect of the invention there is provided a method of screening compounds comprising the step of contacting the compound of interest with the stem cell-derived, in vitro generated, retinal organoid described above.

Optionally the method comprises the step of assaying organoid viability or assaying viability of various retinal cell types within the organoid, wherein an impaired organoid viability or impaired viability of retinal cell types within the organoid determination indicates that said compound of interest is likely to induce a serious adverse event.

Optionally the method comprises the step of assaying organoid viability or assaying viability of various retinal cell types within the organoid, wherein an improved organoid viability or improved viability of retinal cell types within the organoid determination indicates that said compound of interest is likely to have value in the treatment of retinal diseases.

Advantageously assays of functional output can also be carried out as cells may be alive but not functioning in a normal manner.

Optionally the method comprises the step of assaying organoid functional output or assaying the functional output of various retinal cell types within the organoid, wherein an impaired organoid functional output or impaired functional output of retinal cell types within the organoid determination indicates that said compound of interest is likely to induce a serious adverse event.

Optionally the method comprises the step of assaying organoid functional output or assaying functional output of various retinal cell types within the organoid, wherein an improved functional output or improved functional output of retinal cell types within the organoid determination indicates that said compound of interest is likely to have value in the treatment of retinal diseases.

According to another aspect of the present invention there is a method of treating an individual having retinal damage or a retinal disorder, comprising implanting a stem cell-derived, in vitro generated, retinal organoid as described above.

Optionally the retinal disorder is inherited retinal dystrophy, diabetic retinopathy, retinopathy of prematurity, macular degeneration, age-related macular degeneration, Usher syndrome, Stargardt disease or Retinitis Pigmentosa.

Another aspect of the present invention relates to use of the tissue or organoid indicated above in medicine, more particularly for the treatment of retinal disorders, yet more particularly for the treatment of one or more selected from inherited retinal dystrophy, diabetic retinopathy, retinopathy of prematurity, macular degeneration, age-related macular degeneration, Usher syndrome, Stargardt disease or Retinitis Pigmentosa.

Another aspect of the present invention relates to use of the tissue or organoid indicated above in a drug discovery screen; toxicity assay; research of tissue embryology, cell lineages, and differentiation pathways; gene expression studies including recombinant gene expression; research of mechanisms involved in tissue injury and repair; research of inflammatory and infectious diseases; studies of pathogenetic mechanisms; or studies of mechanisms of cell transformation and aetiology of retinal disease.

Various further features and aspects of the invention are defined in the claims.

Unless defined otherwise, all technical and scientific terms used herein have the meaning commonly understood by a person skilled in the art to which this invention belongs.

To assist the reader, the following terms have the meanings ascribed to them below, unless specified otherwise.

The term “organoid” refers to an organised mass of cell types, generated in vitro that mimics at least to some degree the structure, marker expression, or function of a naturally occurring organ.

The term “retinal organoid” refers to organoids which mimic human retinogenesis through formation of organized layered retinal structures that display markers for typical retinal cell types.

The term “detect” refers to identifying the presence, absence or amount of an analyte to be detected.

The term “disease” refers to any condition or disorder that damages or interferes with the normal function of a cell, tissue, or organ. For example, retinal degenerative diseases such as Retinitis Pigmentosa and age-related macular degeneration (AMD) are the major causes of vision loss due to cell death or functional loss of photoreceptor cells (PRCs) and/or retinal pigment epithelium (RPE).

The term “marker” refers to any protein or polynucleotide analyte having an expression level or activity associated with a particular cell type. In one embodiment, transcriptomics can be used to measure the levels of markers associated with cell fate, cell differentiation, and cell specific structure or function.

The terms “treat,” treating,” “treatment,” and the like refer to reducing or ameliorating a disorder and/or symptom(s) associated therewith. It will be appreciated that, although not precluded, treating a disorder or condition does not require that the disorder, condition or symptoms associated therewith be completely eliminated.

The term “pluripotent stem cells (PSCs),” also commonly known as PS cells, encompasses any cells that can differentiate into nearly all cells, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of totipotent cells, derived from embryos (including embryonic germ cells) or obtained through induction of a non-pluripotent cell, such as an adult somatic cell, by forcing the expression of certain genes.

The term “induced pluripotent stem cells (iPSCs),” also commonly abbreviated as IPS cells, refers to a type of pluripotent stem cells artificially derived from a normally non-pluripotent cell, such as an adult somatic cell, by inducing a “forced” expression of certain genes.

The term “agent” means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc.

The term “medium” (also referred to as a “culture medium” or “cell culture medium”) means a medium for culturing cells containing nutrients that maintain cell viability and support proliferation.

The term “expression” refers to the cellular processes involved in producing RNA and proteins and as appropriate, secreting proteins, including where applicable, but not limited to, transcription, translation, folding modification and processing. Expressed markers include RNA transcribed from a gene and polypeptides obtained by translation of mRNA transcribed from a gene.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991).

### BRIEF DESCRIPTION OF THE DRAWINGS

Embodiments of the present invention will now be described by way of example only with reference to the accompanying drawings where like parts are provided with corresponding reference numerals and in which:

FIG. 1B provides images of improved retinal organoids incorporating microglial cells obtained using a method of the present invention where the microglial cells were obtained from iPSCs; FIG. 1A provides images of improved retinal organoids incorporating microglial cells obtained using a method of the present invention where the microglial cells are primary microglial cells.

FIG. 2 is a diagram showing the method steps of the present invention FIG. 2A is a diagram showing the method steps of another embodiment of the present invention

FIG. 3A shows a retinal-like organoid after 18 days of co-culture with microglial cells, where the organoid obtained using the RLOD method was 278 days old when the microglial cells were added. The staining shows Hoechst staining and IBA1 staining and indicates that that microglial cells are predominantly on the surface rather than integrated. FIG. 3B shows a retinal-like organoid in accordance with the invention, where the organoid obtained using the RLOD method had microglia added at 93 days and were cocultured for 14 days before imaging. The staining shows microglial cells integrated into a retinal organoid.

FIG. 4 shows a retinal-like organoid after approximately 55 days of co-culture with hiPSC derived microglial cells.

### DETAILED DESCRIPTION

The inventors have identified that whilst microglia are the primary resident immune cell type in the central nervous system (CNS), they are not present in current retainal-like organoid models. As microglia are thought to play roles in the development of the retina as well as in the survival of retinal neurons an improved retinal organoid would include microglial cells in a manner similar to that found in naturally occurring retina. Furthermore, it has been demonstrated that microglia play a role in various retinal pathologies such as glaucoma, retinitis pigmentosa and age-related macular degeneration.

However, the culture and study of microglia in vitro is challenging. Acquiring and maintaining primary microglial cells is known to be rare and challenging. Furthermore, isolated primary microglia often have a limited proliferative potential. To overcome this, methods of deriving microglia from induced pluripotent stem cells (iPSCs) have been developed (Abud et al., 2017).

The inventors have identified that the ability to examine the relationship between the retinal organoid and microglia in vitro would further the understanding of the development and function of the organ as well as making drug screening and toxicology assays more representative and accurate and have developed methodology which allows the integration of microglial cells into a retinal organoid model.

Methods of Producing an Improved Retinal Organoid with Integrated Microglial Cells

In some embodiments, an important step is to obtain stem cells that are pluripotent or can be induced to become pluripotent. In some embodiments, exemplary iPS cell lines include but not limited to iPS-SB-Ad4; iPS-SB-Ad3; iPS-DF19-9; iPS-DF4-3; iPS-DF6-9; iPS (Foreskin); and iPS(IMR90).

Human induced pluripotent stem cell (iPSC) derived retinal organoids were generated as follows by co-culturing microglial cells with retinal organoid type structures. The methods below include examples of how the microglial cells and retinal organoid structures could be obtained prior to co-culturing, however it would be understood by one skilled in the art that other methods to obtain these materials could be used. For example, it is envisaged that primary microglial cells could also be obtained and used.

Exemplary Microglial Differentiation Protocol (MDP)
1. Plate induced pluripotent stem cells (iPSCs), such as those from the SB-Ad3 cell line, on matrigel-coated plates at a density of 15×10³ cells/cm² in custom mTeSR1 medium (custom mTeSR1 is mTeSR1 without the growth factors: lithium chloride, GABA, pipecolic acid, bFGF, TGFβ1, BMP4; obtainable from Stem cell technologies)+10 μM Rock Inhibitor for 24 hours
2. Feed every day with custom mTeSR1
3. When individual colonies are visible (2-4 days), induce differentiation:
   a. mTeSR custom medium containing 80 ng/ml BMP4
4. Change medium daily
5. On day 4, change medium to:
   a. StemPro-34 serum free medium (SFM) (containing 2 mM GutaMAX-I, Life Technologies)
   b. 25 ng/ml bFGF (basic fibroblast growth factor)
   c. 100 ng/ml SCF (stem cell factor)
   d. 80 ng/ml VEGF (Vascular endothelial growth factor)
6. On day 6, switch medium to:
   a. StemPro-34 SFM serum free medium (containing 2 mM GlutaMAX-I, Life Technologies)
   b. 50 ng/ml SCF (stem cell factor)
   c. 50 ng/ml IL-3 (Interleukin 3)
   d. 5 ng/ml thrombopoietin (TPO)
   e. 50 ng/ml macrophage colony-stimulating factor (M-CSF)
   f. 50 ng/ml fms-like tyrosine kinase 3 (Flt3) ligand
7. On day 10, pellet the supernatant fraction, resuspend in fresh medium and return to the dishes.
8. On day 14, pellet floating cells, resuspend in:
   a. StemPro-34
   b. 50 ng/ml M-CSF
   c. 50 ng/ml Flt3 ligand
   d. 25 ng/ml Granulocyte-macrophage colony-stimulating factor (GM-CSF)
   e. Re-plate the cells back to their dishes
9. Repeat step #8 every 4 days until there are >40% cells positive for CD14 and/or CX3CR1 at which point they could either continue to grow as per point 8 or be re-plated for microglial maturation on tissue culture treated plates.
10. After re-plating CD14+ and/or CX3CR1+ cells change medium to Microglial Medium:
   a. RPMI-1640 growth medium supplemented with 2 mM GlutaMAX-1
   b. 10 ng/ml GM-CSF
   c. 100 ng/mlInterleukin 34 (IL-34)
11. Change medium every 3 to 4 days thereafter

NOTE: Penicillin-Streptomycin (pen/strep) is added at all stages of cell culture

mTeSR1 medium is feeder-free maintenance medium for human ES and iPS Cells.

Exemplary protocol for retinal-like organoid differentiation (RLOD)
1. Day −2:
   a. Wash iPSCs cells with PBS. These could be cells from the SB-Ad4 cell line i.e. a different cell line than used to obtain microglial like cells, but the same iPSC line could also be used.
   b. Add Accutase™ (at room temperature) for 3 mins. Accutase™ being a cell detachment solution of proteolytic and collagenolytic enzymes.
   c. Dilute with mTeSR1+10 uM Rho-associated, coiled-coil containing protein kinase (ROCK) inhibitor
   d. Centrifuge for 3 mins at 1000 rpm
   e. Resuspend in 10 ml mTeSR1+10 uM ROCKi and count
   f. Plate 7000 cells per well of a 96-well plate pre-coated with lipidure in 100 ul mTeSR1+10 uM ROCKi
   g. Do not touch for 48 hours
2. Day 0:
   a. Add 200 ul of differentiation medium and feed every 2 days by half-replacing the medium with 100 ul thereafter
   b. Medium composition
      i. 41% Iscove's Modified Dulbecco's Medium (IMDM)
      ii. 41% HAM's F12 nutrient mixture
      iii. 15% KnockOut™ Serum replacement (KOSR) i.e. a more defined, FBS-free medium supplement that supports the growth of pluripotent stem cells
      iv. 1% GlutaMAX
      v. 1% Chemically defined lipid concentrate
      vi. 1% Pen/Strep
      vii. 225 uM 1-Thioglycerol
3. Day 6:
   a. Add 2.25 nM BMP4
   b. Replace ½ with fresh medium every 3^{rd} day
4. Day 18:
   a. Change medium to reversal medium and feed every 2 days
   b. DMEM/F12 (containing Glutamax)
   c. 1% N2
   d. 4 uM CHIR99021 (an inhibitor of the enzyme GSK-3)
   e. 2.5 uM SU5402 (MEK/ERK pathway inhibitor; Inhibits VEGFR2, FGFR1, and PDGFRB)
   f. 1% Pen/Strep
5. Day 24:
   a. Change medium to maintenance medium
   b. DMEM/F12 (containing Glutamax)
   c. 5% FBS
   d. 1% N2
   e. 0.25 uM Retinoic Acid
   f. 0.1 mM Taurine
   g. 1% Pen/Strep
   h. 0.25 ug/ml Fungizone™ (Amphotericin B)
   i. Feed ×3 per week for 2 months until medium doesn't change colour, then ×2 per week

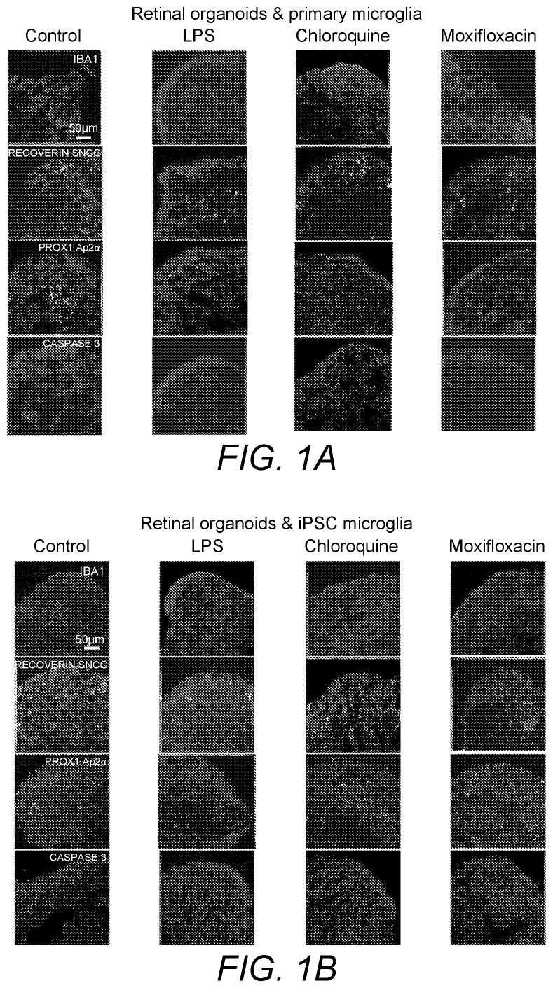

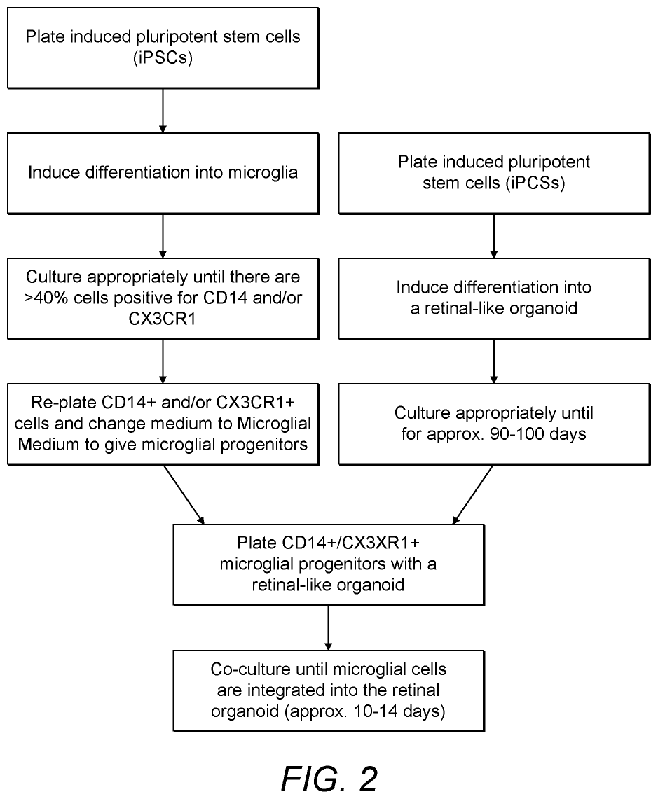

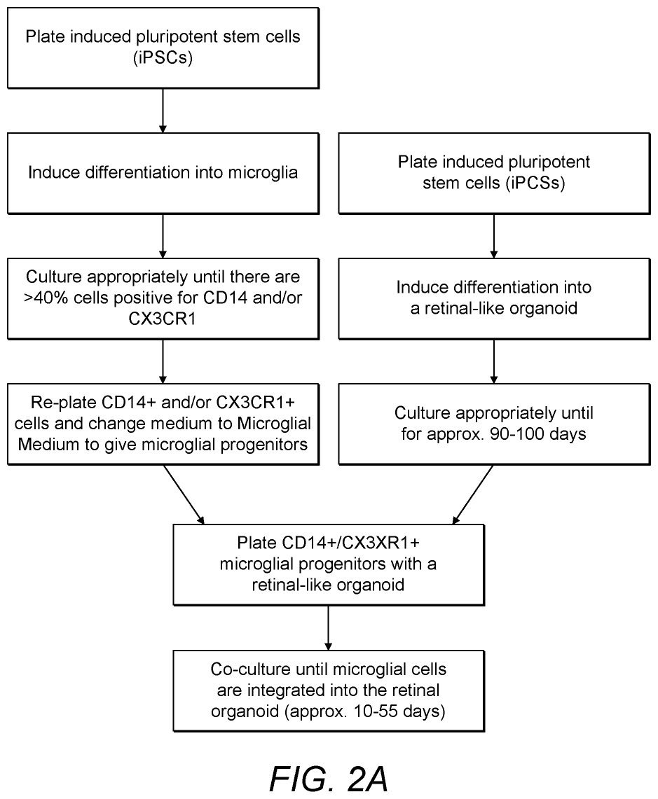

Method for Obtaining an Improved Retinal Organoid Incorporating Microglial Cells
1. CD14+/CX3XR1+ microglial progenitors from supernatant (step 10 of MDP) are plated on tissue culture plates in microglial medium and are matured for 7 days
2. On day 8, cells are dissociated and re-plated in wells with retinal-like organoids (obtained via the RLOD method) at a density of 5000 cells per organoid in the following growth medium:
   a. DMEM/F12 (containing Glutamax)
   b. 5% FBS
   c. 1% N2
   d. 0.25 uM Retinoic Acid
   e. 0.1 mM Taurine
   f. 1% Pen/Strep
   g. 10 ng/ml GM-CSF
   h. 100 ng/ml IL-34
   This is referred to as the co-culture.
3. The co-cultured cells are left to integrate within the organoids for 14 days, feeding them 2 times a week.
4. After 14 days there is an improved retinal organoid which incorporates microglial cells into a layered 3D retina-like structure. Images showing the formation of an improved organoid can be seen in FIG. 1.

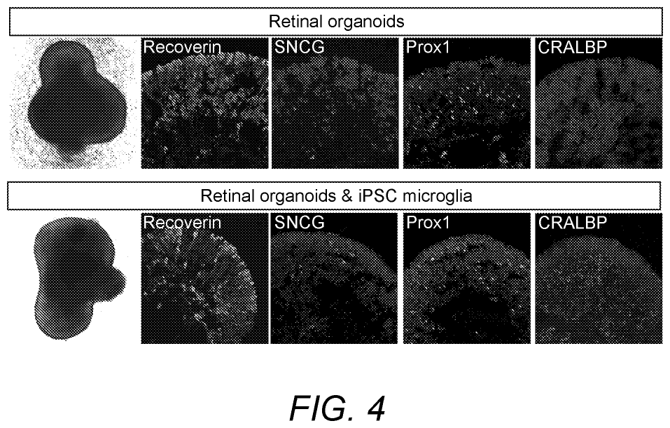

In an alternative method to produce a yet further improved retinal organoid, steps 3 and 4 allow for a longer co-culturing period, such that the method steps are:
1. CD14+/CX3XR1+ microglial progenitors from supernatant (step 10 of MDP) are plated on tissue culture plates in microglial medium and are matured for 7 days
2. On day 8, cells are dissociated and re-plated in wells with retinal-like organoids (obtained via the RLOD method) at a density of 5000 cells per organoid in the following growth medium:
   a. DMEM/F12 (containing Glutamax)
   b. 5% FBS
   c. 1% N2
   d. 0.25 uM Retinoic Acid
   e. 0.1 mM Taurine
   f. 1% Pen/Strep
   g. 10 ng/ml GM-CSF
   h. 100 ng/ml IL-34
   This is referred to as the co-culture.
3. The co-cultured cells are left to integrate within the organoids for 55 days, feeding them 2 times a week.
5. After 55 days there is an improved retinal organoid which incorporates microglial cells into a layered 3D retina-like structure. Images showing the formation of an improved organoid can be seen in FIG. 4.

It has been found that it is preferable to start the co-culture using microglial cells that are left to mature for 7 days, rather than taking monocytes directly from supernatant (day 9 MDP). It is surmised that the progenitor population is enriched by leaving them in their maturation medium for an extra 7 days.

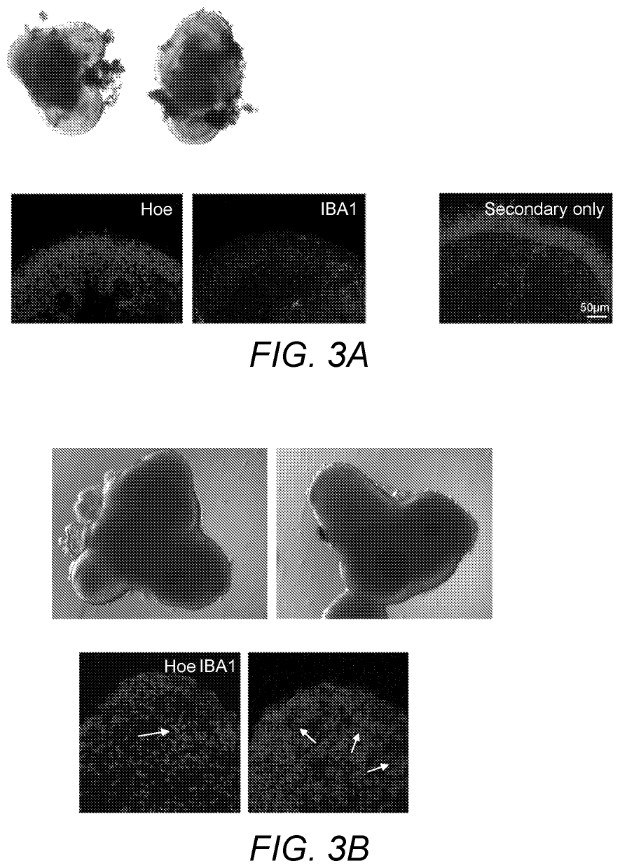

It is also preferred to co-culture with organoids <250 days old, more preferably <200 days old and yet more preferably <150 days old. For example, examples of tests using organoids at 202 and 267 of retinal differentiation yielded less desirable products. However, good results were obtained well with organoids that were ˜90-100 days old. FIG. 3 shows a retinal-like organoid where the organoid obtained using the RLOD was 278 days old when the microglial cells were added. The bright-field image shows the microglia is sitting on top of the organoid and the IHC image with Hoe and IBA1 confirms that there were no cells that integrated.

Preferably the microglial cells are plated at a density of <10000 cells per organoid. Most preferably the microglial cells are plated at a density of <8000 cells per organoid. Preferably the microglial cells are plated at a density of approximately 5000 cells per organoid.

Whilst the preferred embodiment utilises microglial cells obtained from iPSCs, it is possible to also use primary microglial cells. Although challenging, it is possible to obtain primary cells, for example from adult CNS tissue, using known techniques.

The starting iPSCs can be selected to provide an organoid from a healthy individual. It is of course also possible to use cells of patients with a given mutation, inducing pluripotent stem cell status and performing the inventive methods to induce tissue development as described above to provide a model organoid for a particular disease state or states.

Polypeptide or polynucleotide expression of cells within the organoid or the constituent tissues can be determined and/or compared by procedures well known in the art, such as western blotting, flow cytometry, immunocytochemistry, in situ hybridization, fluorescence in situ hybridization (FISH), ELISA, microarray analysis, RT-PCR, Northern blotting, or colorimetric assays, such as the Bradford Assay and Lowry Assay.

For example, in the MDP method steps described above, CD14/CX3CR1 could be tested by flow cytometry, and in variations where IBA1 is also checked this could be done by immunofluorescence.

The present invention further provides a method of screening a candidate agent to determine suitability for treating a retinal tissue defect of interest, comprising administering the candidate agent to the organoid of the present invention (i.e. organoids with both 3-dimensional retinal cells and integrated microglial cells) obtained using the methods according to the present invention, and determining the effect on the organoid. Whilst it is envisaged that candidate agents would be administered to the organoid it is also conceivable that the agents could be incorporated during the production of the organoid to understand the effects on the development of said organoid. According to this aspect, a candidate agent, e.g., a candidate therapeutic drug, can be screened for having an effect on organoids which have a known mutation, which can be introduced as described above, in particular, the present invention provides investigations in mutations in microcephaly and allows the screening of pharmaceutical agents, which can affect the mutations, e.g. compensate for the insufficiency or overexpression in the mutated gene. A positive candidate drug could be a compound, which restores normal cellular development, as can be observed by performing the inventive tissue generation method without a mutation for comparison, e.g. by using healthy pluripotent stem cells.

Of course, it is also possible to screen candidate agents, e.g. candidate therapeutic drugs, to have any effect on normal tissue as well, without a mutation, which leads to an aberrant development. Thus in yet another aspect, the invention relates to a method of testing a candidate drug for physiological effects, comprising administering a candidate drug to an artificial culture and determining an activity of interest of the cells of said culture and comparing said activity to an activity of cells to the culture without administering said candidate drug, wherein a differential activity indicates an effect.

The present invention also envisages that the organoids, or a cell derived from said organoids, could be used in a drug discovery screen; toxicity assay; research of tissue embryology, cell lineages, and differentiation pathways; gene expression studies including recombinant gene expression; research of mechanisms involved in tissue injury and repair; research of inflammatory and infectious diseases; studies of pathogenetic mechanisms; or studies of mechanisms of cell transformation and aetiology of retinal disease.

The organoid of the invention, or a cell derived from said organoid, is also envisaged for use in medicine. For example, said organoid, or a cell derived from said organoid, could be used for use in treating a retinal disorder, condition or disease such as diabetic retinopathy, retinopathy of prematurity, macular degeneration, Usher syndrome, Stargardt disease, Retinitis Pigmentosa, age-related macular degeneration (AMD) and inherited retinal dystrophies (HRDs). One option is that said organoid, or a cell derived from said organoid could be used in regenerative medicine, for example, wherein the use involves transplantation of the organoid or cell into a patient.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” should be interpreted as “including but not limited to,” the term “having” should be interpreted as “having at least,” the term “includes” should be interpreted as “includes but is not limited to,”, the term “comprising/comprises” should be interpreted as “comprising/comprises but is not limited to” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

It will be appreciated that various embodiments of the present disclosure have been described herein for purposes of illustration, and that various modifications may be made without departing from the scope of the present disclosure. Accordingly, the various embodiments disclosed herein are not intended to be limiting, with the true scope being indicated by the following claims.

1. A pluripotent stem cell-derived, in vitro generated, retinal tissue comprising retinal cells and microglial cells.

2. The pluripotent stem cell-derived, in vitro generated, retinal tissue of claim 1 which is a three-dimensional retinal-like organoid.

3. The pluripotent stem cell-derived, in vitro generated, retinal tissue of claim 2 wherein the three-dimensional retinal-like organoid comprises microglial cells, an optional retinal pigment epithelium (RPE) layer, and a plurality of cell types selected from photoreceptor cells (PRCs), amacrine cells, muller glia, horizontal cells, bipolar cells and retinal ganglion cells.

4. The pluripotent stem cell-derived, in vitro generated, retinal tissue of claim 1 which comprises an induced pluripotent stem cell-derived, in vitro generated, retinal tissue.

5. The induced pluripotent stem cell-derived, in vitro generated, retinal tissue of claim 4 comprising induced pluripotent stem cells (iPSCs) derived from a patient without any known genetic disorder.

6. The induced pluripotent stem cell-derived, in vitro generated, retinal tissue of claim 4 comprising induced pluripotent stem cells (iPSCs) derived from a patient with a known genetic disorder.

7. The pluripotent stem cell-derived, in vitro generated, three-dimensional retinal-like organoid of claim 2 wherein the organoid comprises a cell expressing one or more retinal cell markers and a cell expressing one or more microglial markers.

8. The pluripotent stem cell-derived, in vitro generated, three-dimensional retinal-like organoid of claim 7 wherein the retinal cell markers comprise AP-2α, HuC/D, Prox1, Recoverin and/or CRX.

9. The pluripotent stem cell-derived, in vitro generated, three-dimensional retinal-like organoid of claim 7 wherein the expressed microglial cell markers comprise CD14 and/or CX3C chemokine receptor 1 (CX3CR1) and/or Ionized calcium binding adaptor molecule 1 (IBA1).

10. A method for obtaining a retinal tissue or a retinal organoid comprising co-culturing microglial or microglial progenitor cells and the retinal organoid under conditions which allow the microglial cells to integrate into the organoid.

11. The method for obtaining the retinal tissue or the retinal organoid of claim 10, the method comprising:
(i) obtaining a population of microglial cells and/or microglial progenitor cells;
(ii) obtaining a pluripotent stem cell derived retinal organoid; and
(iii) co-culturing the microglial cells and the retinal organoid under conditions which allow the microglial cells to integrate into the organoid.

12. The method for obtaining the retinal tissue or the retinal organoid of claim 11 wherein the obtained population of microglial or microglial progenitor cells has >40% cells positive for CD14 and/or CX3CR1.

13. The method for obtaining the retinal tissue or the retinal organoid of claim 11 wherein obtaining the population of microglial cells further comprises differentiating pluripotent stem cells into hematopoietic progenitor cells and then to microglial progenitors or microglial-like cells.

14. The method for obtaining the retinal tissue or the retinal organoid of claim 11 wherein the population of microglial cells or microglial progenitor cells comprises induced pluripotent stem cell derived microglial cells.

15. The method for obtaining the retinal tissue or the retinal organoid of claim 11 wherein the retinal tissue or organoid has a layered structure resembling a naturally occurring retina and comprises microglial cells, an optional retinal pigment epithelium (RPE) layer, and a plurality of cell types selected from photoreceptor cells (PRCs), amacrine cells, muller glia, horizontal cells, bipolar cells and retinal ganglion cells.

16. The method for obtaining the retinal tissue or the retinal organoid of claim 10 wherein the retinal tissue or organoid is positive for AP-2α, HuC/D, Prox1, Recoverin and/or CRX.

17. The method for obtaining the retinal tissue or the retinal organoid of claim 10 wherein, prior to co-culturing the microglial cells and the retinal tissue or the retinal organoid, the microglial cells are left to mature for 3 or more days.

18. The method for obtaining the retinal tissue or the retinal organoid of claim 10 wherein, when co-culturing the microglial cells and the retinal tissue or the retinal organoid, the organoid is <250 days old.

19. The method for obtaining the retinal tissue or the retinal organoid of claim 10 wherein, when co-culturing the microglial cells and the retinal tissue or the retinal organoid, the microglial cells are plated at a density of <10000 cells per organoid.

20. The method for obtaining the retinal tissue or the retinal organoid of claim 10 wherein the microglial cells and the retinal tissue or the retinal organoid are co-cultured for greater than 7 days.

21. (canceled)

22. (canceled)

23. (canceled)

24. (canceled)

25. (canceled)

26. The method for obtaining the retinal tissue or the retinal organoid of claim 10 wherein, prior to co-culturing the microglial cells and the retinal tissue or organoid, the microglial cells are left to mature for 5 or more days.

27. The method for obtaining the retinal tissue or the retinal organoid of claim 10 wherein, prior to co-culturing the microglial cells and the retinal tissue or the retinal organoid, the microglial cells are left to mature for 7 or more days.

28. The method for obtaining the retinal tissue or the retinal organoid of claim 10 wherein, when co-culturing the microglial cells and the retinal tissue or the retinal organoid, the organoid is <200 days old.

29. The method for obtaining the retinal tissue or organoid of claim 10 wherein, when co-culturing the microglial cells and the retinal tissue or the retinal organoid, the organoid is <150 days old.

30. The method for obtaining the retinal tissue or organoid of claim 10 wherein, when co-culturing the microglial cells and the retinal tissue or the retinal organoid, the microglial cells are plated at a density of <8000 cells per organoid.

31. The method for obtaining the retinal tissue or the retinal organoid of claim 10 wherein the microglial cells and the retinal tissue or the retinal organoid are co-cultured for greater than 10 days.

32. The method for obtaining the retinal tissue or the retinal organoid of claim 10 wherein the microglial cells and the retinal tissue or the retinal organoid are co-cultured for greater than 14 days.

33. The method for obtaining the retinal tissue or the retinal organoid of claim 10 wherein the microglial cells and the retinal tissue or the retinal organoid are co-cultured for greater than 55 days.
